(19) United States
(12) Patent Application Publication
(10) Pub. No.: US 2018/0043102 A1
(43) Pub. Date: Feb. 15, 2018

(54) O-Ring Plunger for a Prefilled Syringe and Method

(71) Applicant: Becton, Dickinson and Company, Franklin Lakes, NJ (US)

(72) Inventors: Gheorghe Cojocariu, Bridgewater, NJ (US); Kevin M. Ryan, Whitehouse Station, NJ (US)

(21) Appl. No.: 15/232,233

(22) Filed: Aug. 9, 2016

(51) Int. Cl. A61M 5/315

(52) U.S. Cl. CPC A61M 5/31515; A61M 5/31513; A61M 2207/00

(57) ABSTRACT

A multi-piece plunger configured to be slideably advanced through a syringe barrel to expel fluid therefrom is provided. The plunger includes a head portion having a proximal end, a distal end, and an annular sidewall extending therebetween. The sidewall can define at least two annular grooves. The plunger also includes annular seals disposed within each of the at least two annular grooves, such that at least a portion of each of the annular seals protrudes radially beyond an outer surface of the sidewall to form a slideable seal with an inner sidewall of the syringe barrel. The head portion can be formed from a first material, such as a rigid plastic material, and the annular seals can be formed from a second material, such as a thermoset rubber or a thermoplastic elastomer. A prefilled syringe including the multi-piece plunger is also provided herein.

### BACKGROUND OF THE INVENTION

### Field of the Invention

The invention relates, in general, to a plunger for use with a syringe, such as a prefilled syringe containing a therapeutic agent to be injected to a patient, and, more particularly, to a multi-piece plunger including a head portion and one or more elastomeric annular seals.

### Description of Related Art

Current syringe designs generally include a tubular barrel having an access opening formed at one end, and a smaller discharge opening (e.g., a nozzle) formed at the opposing end. A lead end of an elongated plunger is received within the access opening of the barrel so as to be slidable within the barrel. Attached to or integral with the lead end of the plunger is a flexible sealing member or stopper that snugly seals against the interior surface of the barrel. A needle, a threaded member, or a non-threaded member is usually attached to the discharge opening on the barrel. The needle can be used to penetrate a surface while the threaded member can be used to attach the syringe to another medical device, such as a catheter. The flexible stopper is usually manufactured from an elastomeric material, such as a rubber or a cross-linked or thermoplastic elastomer.

During use, the discharge end of the syringe is initially placed in contact with a fluid. For example, the needle on the syringe can be inserted into a source of liquid medication such as a medical vial. As the plunger is retracted within the barrel, a process known as aspiration, a negative pressure is formed within the end of the barrel so as to cause the fluid to be drawn into the barrel. The syringe can then be moved to a second location where advancing the plunger within the barrel causes the fluid to be pushed or expressed out the discharge end of the barrel. In some examples, syringes are provided prefilled with a fluid to be administered to a patient and, accordingly, fluid does not need to be drawn into the syringe barrel immediately prior to use.

Conventional elastomeric plungers and stoppers generally include an open proximal end configured to engage a plunger rod, a distal surface, often referred to as a roof, configured to come into contact with the fluid contained in the syringe barrel, and a sidewall extending therebetween. The sidewall can include protrusions, such as ribs, fins, or flanges, for creating variable contact with the inner surface of the syringe barrel. Due to the high contact forces and high friction coefficient between the protrusions and the inner wall of the barrels, current elastomeric plunger designs are often lubricated with a liquid lubricant, such as silicone oil, so that break-loose and break-out forces required to operate the syringe are not too high. An exemplary method for coating a sealing member with a lubricant by tumbling is disclosed in U.S. Pat. No. 7,141,042, entitled “Low silicone glass prefillable syringe”, which is incorporated by reference herein in its entirety. Elastomeric stoppers formed, for example, by injection molding can be tumbled and/or immersed in a silicone bath thereby covering the entire surface of the plunger or stopper with the lubricant. Since only the protrusions or ribs of the plunger sidewall actually contact the syringe barrel, a large portion of the surface area of the coated or lubricated plunger or stopper does not actually contribute to the sliding properties of the syringe and stopper. As such, the amount of lubricant required for each plunger and associated manufacturing costs could be reduced if only the interface between the plunger and syringe barrel were lubricated.

Current rubber stoppers are often manufactured from a specialized cross-linked rubber (e.g., polyisoprene) that requires a specialized compression molding/curing process. Such a process can significantly increase the cost of manufacturing the syringe. Furthermore, significant waste materials are also produced during the compression molding process. For example, in a typical manufacturing process, up to 30% of the rubber is discarded during manufacturing of the conventional elastomeric stopper.

For these reasons, a need exists for a stopper that can be efficiently manufactured, thereby reducing manufacturing costs and the amount of waste materials, including elastomeric materials and lubricants, produced during the manufacturing of the syringe. The plunger and syringe assembly disclosed herein are designed to address these issues.

### SUMMARY OF THE INVENTION

According to an aspect of the disclosure, a plunger configured to be slidably advanced through a syringe barrel to expel fluid therefrom is provided. The plunger includes a head portion having a proximal end, a distal end, and an annular sidewall extending therebetween. The sidewall can define a first annular groove and a second annular groove. The plunger also includes a first annular seal disposed within the first annular groove, and a second annular seal disposed within the second annular groove, such that at least a portion of each of the annular seals protrudes radially beyond an outer surface of the sidewall to form a slideable seal with an inner sidewall of the syringe barrel.

In some examples, the head portion can include a first material, and the annular seals can include a second material that is different from the first material. For example, the first material can include one or more of polypropylene, polyethylene, cyclic olefin polymer or copolymer, polycarbonate, and polyester. The second material can include one or more of a thermoset rubber and a thermoplastic elastomer. The thermoset rubber can be butyl rubber, styrene-butadiene rubber (SBR), and/or poly-isoprene rubber. The thermoplastic elastomer can be one or more of thermoplastic olefins, styrenic block copolymers, thermoplastic polyurethanes, and thermoplastic polyamides.

In some examples, the annular seals can include a lubricant disposed on an outer surface thereof. The lubricant can be silicone. The head portion of the plunger can be lubricant free.

In some examples, the head portion defines a cavity configured to receive a receiving portion of a plunger rod. The cavity may extend inwardly from the proximal end of the plunger head. The cavity can include a sidewall defining a plurality of threaded grooves configured to engage corresponding threads of the distal end of the plunger rod. The head portion may be configured to receive a portion plunger rod in a snap-fit configuration. The cavity may also include one or more longitudinal supports extending in a distal direction from a distal end thereof. The longitudinal supports can be configured to support the closed distal end of the head portion. The distal end of the head portion can be a conical contact surface.

In some examples, the at least two annular grooves are spaced apart in a longitudinal direction by a gap having a length that is greater than or equal to a thickness of the annular seals. In other examples the gap may have a length that is smaller than the thickness of the annular seals. The at least two annular grooves can be a concavely curved surface having a curvature that corresponds to the curvature of the annular seals.

According to another aspect of the disclosure, a syringe is provided. The syringe includes a substantially cylindrical barrel having an open proximal end, a distal end including a nozzle, and a sidewall extending therebetween. The syringe also includes a plunger slideably disposed within the syringe barrel and configured to be advanced through the barrel in a distal direction to expel fluid through the nozzle. The plunger can include a head portion including a proximal end, a distal end, and an annular sidewall extending therebetween. The sidewall can define at least two annular grooves. The plunger further includes annular seals disposed within each of the at least two annular grooves, such that at least a portion of each of the annular seals protrudes radially beyond the sidewall to form a slideable seal with an inner sidewall of the syringe barrel.

In some examples, the syringe further includes a plunger rod. A distal end of the plunger rod can be removeably inserted in a cavity extending inward from the proximal end of the plunger head. The distal end of the plunger rod may include threaded grooves configured to engage corresponding threaded grooves on an inner sidewall of the cavity. The plunger rod and the head portion may include a first material and the annular seals may include a second material different than the first material. For example, the first material can be one or more of polypropylene, polyethylene, cyclic olefin polymer or copolymer, polycarbonate, and polyester. The second material can be one or more of butyl rubber, styrene-butadiene rubber, poly-isoprene rubber, thermoplastic olefins, styrenic block copolymers, thermoplastic polyurethanes, and thermoplastic polyamides.

According to another aspect of the disclosure, a manufacture of a prefilled syringe is provided. The method includes molding a head portion of a multi-piece plunger, wherein the head portion includes a proximal end, a distal end, and an annular sidewall extending therebetween. The method also includes providing at least one annular seal around at least a portion of the sidewall of the plunger, thereby forming the multi-piece plunger. The method further includes inserting the multi-piece plunger into a syringe barrel thereby forming a prefilled syringe. In some instances, when inserted in the barrel, at least a portion of the at least one annular seal contacts an inner sidewall of the syringe barrel to form a slideable seal therewith.

In some examples, the head portion of the plunger includes a first material and the at least one annular seal includes a second material that is different from the first material. For example, the first material can be one or more of polypropylene, polyethylene, cyclic olefin polymer or copolymer, polycarbonate, and polyester. The second material can be one or more of butyl rubber, thermoplastic olefins, styrenic block copolymers, thermoplastic polyurethanes, and thermoplastic polyamides.

In some examples, providing the at least one annular seal can include forming the annular seal by overmolding the seal on the head portion. In other examples, providing the at least one annular seal can include molding the at least one annular seal from an elastomeric material different from the material used to mold the head portion and assembling the plunger by inserting the at least one annular seal in a groove on the sidewall of the head portion.

In some examples, the method also includes filling the syringe barrel with a fluid prior to insertion of the multi-piece plunger. Inserting the plunger into the syringe barrel can include inserting the plunger by a vented placement method.

According to another aspect of the disclosure, a plunger configured to be slideably advanced through a syringe barrel to expel fluid therefrom is provided. The plunger includes a head portion having a proximal end, a distal end, and an annular sidewall extending therebetween and an annular seal disposed about at least a portion of the sidewall. The annular seal can include at least two annular ribs protruding radially outward from the seal and configured to form a slideable seal with an inner sidewall of the syringe barrel.

In some examples, the head portion can include a first material and the annular seal can include a second material that is different from the first material. The first material can be one or more of polypropylene, polyethylene, cyclic olefin polymer or copolymer, polycarbonate, and polyester. The second material can be one or more of butyl rubber, styrene-butadiene rubber, poly-isoprene rubber, thermoplastic olefins, styrenic block copolymers, thermoplastic polyurethanes, and thermoplastic polyamides.

In some examples, the head portion can include at least two annular grooves on the sidewall thereof. A portion of the annular seal can be disposed within each of the at least two annular grooves. The at least two annular ribs may have a polygonal shaped cross section. In other examples, the at least two annular ribs can be convexly curved.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

FIG. 1 is a perspective view of a multi-piece plunger according to an aspect of the disclosure;

FIG. 2 is a perspective cross-sectional view of the plunger of FIG. 1 taken along line A-A in FIG. 1;

FIG. 3A is a side view of a head portion of the plunger of FIG. 1;

FIG. 3B is a top view of the head portion of FIG. 3A;

FIG. 4A is a top view of an annular seal of the plunger of FIG. 1;

FIG. 4B is a side view of the annular seal of FIG. 4A;

FIG. 5A is a cross-sectional view of the plunger of FIG. 1 prior to insertion in a syringe barrel;

FIG. 5B is a cross-sectional view of the plunger of FIG. 1 inserted in a syringe barrel;

FIG. 6 is a schematic drawings of a syringe assembly including the plunger of FIG. 1 according to an aspect of the disclosure;

FIG. 7 is a perspective cross-sectional view of another example of a multi-piece plunger according to another aspect of the disclosure;

FIG. 8 is a perspective cross-sectional view of another example of a multi-piece plunger according to an aspect of the disclosure;

FIG. 9 is a perspective cross-sectional view of another example of a multi-piece plunger according to an aspect of the disclosure;

FIG. 10 is a perspective cross-sectional view of another example of a multi-piece plunger according to an aspect of the disclosure;

FIG. 11 is a perspective cross section view of an example of a two-piece plunger according to an aspect of the disclosure;

FIG. 12 is a perspective cross-sectional view of another example of a two-piece plunger according to an aspect of the disclosure;

FIG. 13 is a perspective cross-sectional view of another example of a two-piece plunger according to an aspect of the disclosure; and

FIG. 14 is a flow chart illustrating a method of manufacture of a prefilled syringe according to an aspect of the disclosure.

### DETAILED DESCRIPTION OF THE INVENTION

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. The term “proximal” is used to refer to a portion of a device or object that is closest to or manipulated by a user. The term “distal” refers to the portion of the device or object farthest away from the portion manipulated by a user. For example, the end of a syringe grasped by a user is the proximal end, and fluid is expelled from the distal end of the syringe. However, it is to be understood that the invention may assume various alternative variations except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

According to an aspect of the disclosure, a multi-piece plunger is discussed herein. The multi-piece plunger can be configured to be slideably advanced through a substantially tubular structure, such as a syringe barrel, to expel fluid therefrom. In some examples, the plunger includes a head or head portion formed from a substantially rigid plastic material, such as one or more of polyolefins (e.g., polyethylene (PE), polypropylene (PP), and their copolymers), cyclic olefin polymers or copolymers, polyamides (e.g., nylons), polyesters (e.g., PET), polystyrene, polyurethane, polycarbonate, acrylonitrile-butadiene-styrene, fluoropolymers, ionomers, and polyacrylates. The plunger can also include a plurality of annular seals disposed around the head portion. For example, the annular seals can be O-rings having a substantially circular cross section. The annular seals can be formed from an elastomeric material, such as rubber or a thermoplastic elastomer. For example, the annular seals can be formed from one or more of thermoplastic olefins, styrenic block copolymers, thermoplastic polyurethanes, and thermoplastic polyamides. Optionally, the annular seals may be formed from one or more of butyl rubber, styrene-butadiene rubber, poly-isoprene rubber, thermoplastic olefins, styrenic block copolymers, thermoplastic polyurethanes, and thermoplastic polyamides.

Desirably, material and manufacturing costs for the multi-piece plunger are lower than costs associated with conventional elastomeric stoppers and plungers. For example, a multi-piece plunger having a plastic head or head portion and elastomeric annular seals can include about 80% less elastomeric material (by weight) compared to a similarly sized conventional elastomeric plunger. Since elastomeric materials are more expensive than rigid plastics, such as polypropylene, reduction in an amount of elastomeric material in the plunger reduces manufacturing costs. However, since the seal between the plunger and syringe barrel is formed by the elastomeric annular seals, it is believed that the multi-piece plunger provides an improved or at least comparable seal quality and stability compared to conventional elastomeric plungers. Accordingly, the multi-piece plunger meets functional requirements for use in prefilled syringes in a satisfactory manner but with reduced cost compared to conventional elastomeric plungers.

In some examples, the multi-piece plunger is configured to form at least two annular seals with the syringe barrel. Forming multiple annular seals offers a number of benefits compared with stoppers and plungers that only form a single seal with the barrel. For example, a plunger which provides multiple sealing points of contact with the syringe barrel provides a more robust seal, comparable to seals formed from conventional elastomeric stoppers with two or more protrusions or ribs. A seal formed from two or more annular seals is more stable and less susceptible to side loads created as the plunger is advanced or retracted through the syringe barrel, compared to stoppers with a single annular seal. The presence of multiple annular seals also provides a larger sealing area with the barrel which, in conjunction with barrel retaining disks or rings on the plunger rod, acts to prevent loss of sterility when the syringe plunger is pulled back beyond the storage position (e.g., to prevent break-loose contamination). The presence of two or more annular seals or O-rings can also ensure that the multi-piece plunger does not inadvertently invert during insertion into the syringe barrel, as may occur with a plunger having only a single annular seal or point of contact with the syringe barrel.

It is believed that the multi-piece plunger disclosed herein is particularly suitable for use in prefilled syringes due to the quality of the seal between the plunger and syringe barrel and material properties of the head portion and annular seals. For example, the multi-piece plunger can be configured to be inserted in a syringe barrel by conventional high speed assembly techniques used with conventional elastomeric stoppers. Accordingly, prefilled syringes having multi-piece plungers can be manufactured rapidly in high speed production facilities. High speed assembly often involves a vented insertion process in which the syringe is filled with a selected fluid through the open proximal end of the syringe barrel. After filling, stoppering with the multi-piece plunger is performed by a vented placement process, which permits faster manufacturing speeds with a smaller footprint compared to other techniques, such as tip-filling and vacuum stoppering. Vented placement of the stopper or plunger generally involves inserting the plunger in a tube or brace having an outer diameter that is slightly smaller than the inner diameter of the syringe barrel and an inner diameter that is slightly larger than the diameter of the head portion of the plunger. The multi-piece plunger is contracted when inserted in the tube or brace. The tube or brace and contracted plunger are inserted to the barrel and, once in a desired position, the tube or brace is removed. Once the tube or brace is removed, the annular seals of the multi-piece plunger expand radially outward to contact the syringe barrel, thereby forming a fluid tight seal between the plunger and barrel.

Since the contact surface or roof of the multi-piece plunger is formed from the hard plastic material, the multi-piece plunger also reduces the amount of elastomer or rubber material that contacts the solution (e.g., therapeutic agent) compared with conventional elastomeric plungers and stoppers. This result is desirable since rubbers tend to have more complex formulations including a larger number of additives and agents (e.g., curing agents) compared with rigid polymers, such as polypropylene or polystyrene. The greater number of additives and agents may increase the likelihood that leachables and extractables will pass from the plunger to the fluid solution over time. Contamination concern is heighted for prefilled syringes in which the fluid is in contact with the plunger for substantial and/or indefinite periods of time. In order to account for the increased likelihood of contamination in prefilled syringes, conventional elastomeric plungers and stoppers are often coated with expensive surface coatings to protect the prefilled solution (drug, biomolecule, etc.), from the rubber material. Coating the stopper with such barrier coatings further increases manufacturing costs for prefilled syringes including conventional elastomeric stoppers. Since the multi-piece plunger disclosed herein replaces the rubber front or roof of the stopper with a more inert rigid plastic material, which is less prone to producing leachables and extractables, such protective coatings may not be needed for multi-piece plungers.

In some examples, the multi-piece plunger can also be designed to reduce an amount of lubrication needed to advance or retract the plunger through the syringe barrel. For example, conventional elastomeric stoppers are often coated and/or immersed in a lubricant, such as silicone oil, to effectively lubricate the interface between radially extending ribs of the plunger and the syringe barrel. While lubricant free stoppers are also known, such lubricant free stoppers may be unsuitable for use with prefilled syringes. Often, the lubrication process effectively applies lubricant to an entire surface of the plunger including the roof or contact surface, meaning that the lubricant comes into contact with the solution contained in the syringe. The contact of stopper lubricants with the solution can have adverse effects for some drugs and biomolecules used as prefilled solutions. However, for the multi-piece stopper, only the annular seals (e.g., O-rings) need to be subjected to the lubrication process. The head or head portion does not need to be lubricated since it does not contact or seal the syringe barrel. Since the head or head portion is not lubricated, the lubricated surface area in contact with the solution is substantially reduced. In addition, reducing the amount of lubricant needed for each syringe further reduces manufacturing costs compared with conventional elastomeric stoppers and plungers.

In some examples, the multi-piece plunger can be designed to demonstrate improved barrier properties compared with elastomeric stoppers and plungers. For example, rigid polymer materials, such as polypropylene, which form the head or head portion of the multi-piece plunger generally form better barriers to oxygen and water vapor compared to soft elastomers. Accordingly, the multi-piece plunger, which replaces a major portion of the elastomer or rubber with rigid plastic, may be expected to form an improved barrier for the prefilled syringe which may prevent contamination and/or degradation of the fluid solution (e.g., the therapeutic agent) and improve shelf life for therapeutic agents and materials in prefilled syringes.

In some examples, the multi-piece plunger disclosed herein also offers certain structural advantages compared with conventional stoppers and plungers. For example, elastomers and rubbers are elastic and characterized by high resilience (e.g., the ability to bounce back after a stress is released). In some instances, the bounce back of a conventional rubber plunger or stopper after the pressure on the plunger (e.g., pressure exerted by a plunger rod when expelling fluid from the syringe) is released, can cause fluid to be drawn back into the syringe barrel after the injection is performed. Such a bounce back is referred to as syringe reflux, in which the plunger bounces backwards (e.g., in a proximal direction away from the end of the syringe) thereby drawing patient fluid (e.g., blood) back into the syringe barrel. However, rigid plastics that form the head or head portion of the multi-piece plunger are not as resilient as elastomeric materials. Therefore, replacing the flexible rubber plunger with a more rigid plastic roof may reduce plunger bounce back and syringe reflux.

In some examples, the multi-piece plunger can be configured to be connected to a plunger rod for advancing and/or retracting the multi-piece plunger through the syringe barrel. The plunger rod can be formed from the same rigid plastic material as the head portion of the multi-piece plunger. The plunger rod can be attached to the multi-piece plunger by, for example, a snap mechanism or threaded connection. The plunger rod can include one or more retaining disks for preventing syringe pull out, as can occur when the multi-piece plunger is retracted through the syringe barrel. These and other features of the multi-piece plunger and prefilled syringe will be discussed in detail herein, in connection with the following examples.

### Exemplary Multi-Piece Plunger

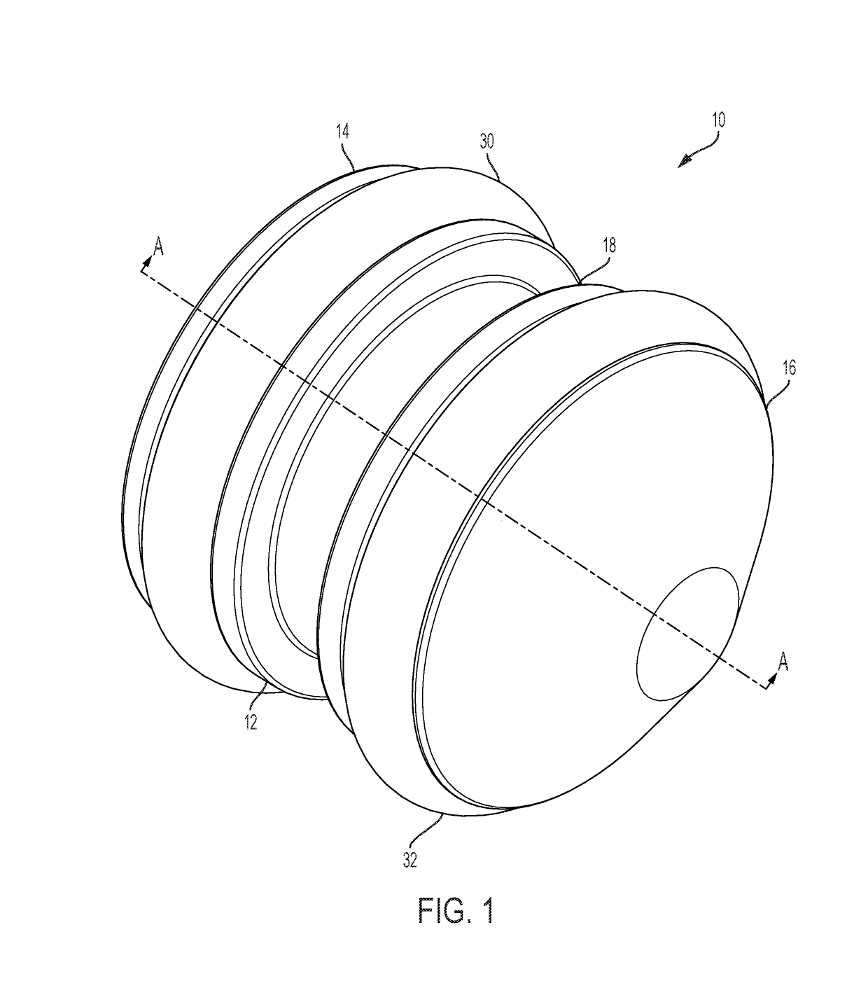

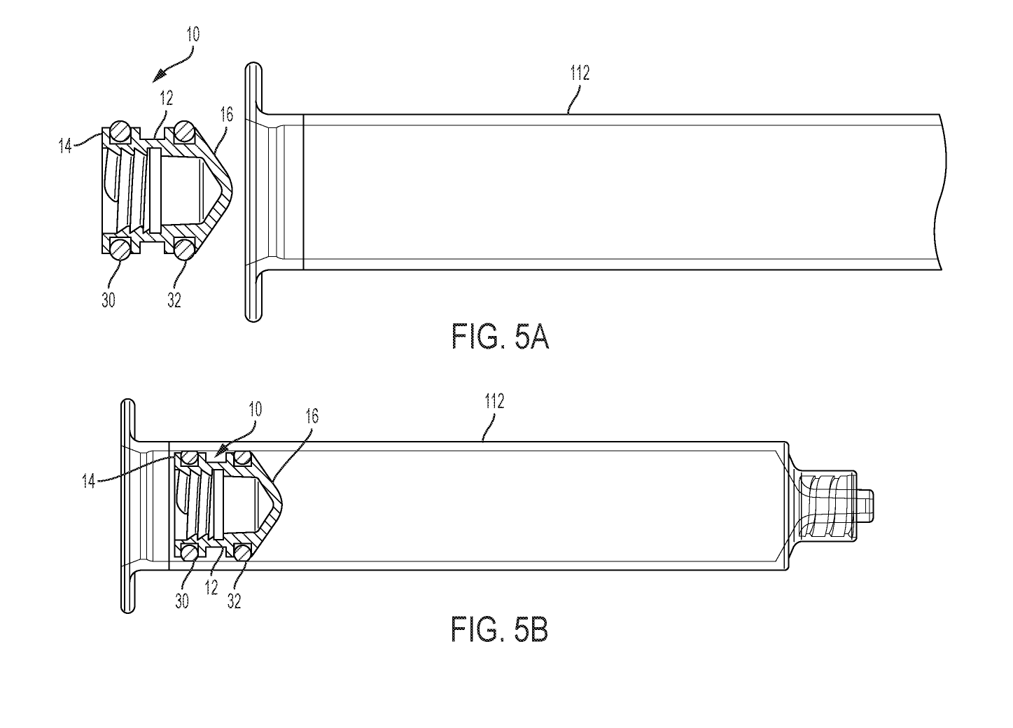

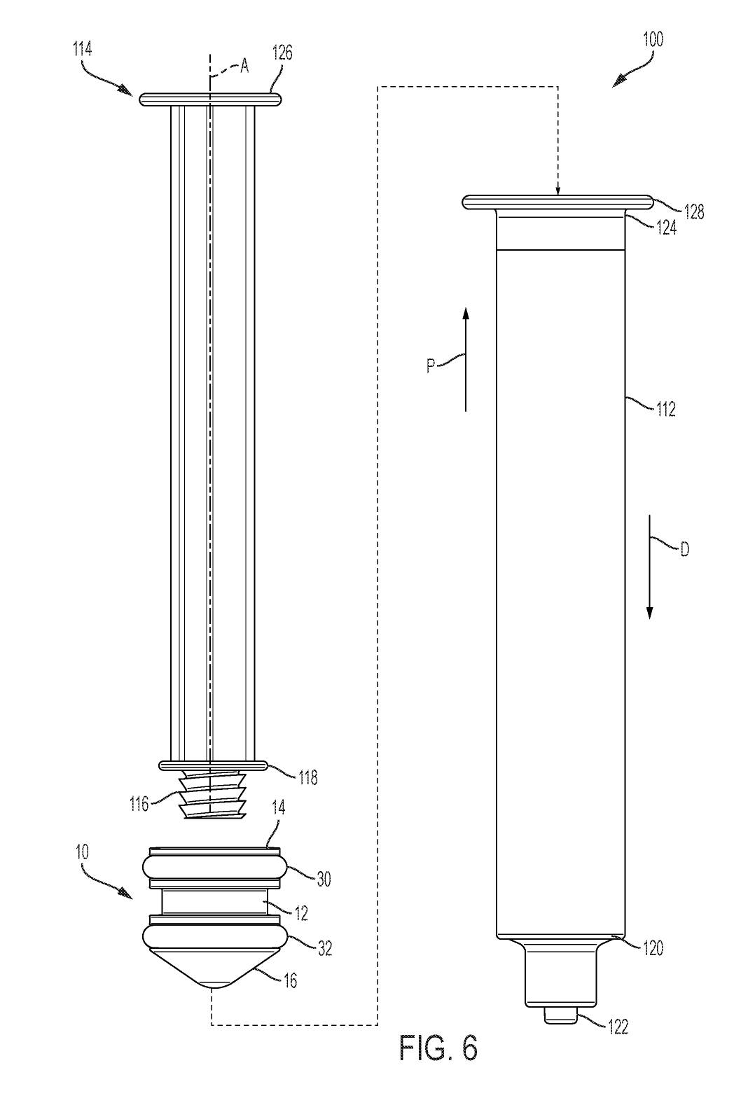

With reference to FIGS. 1, 2, and 5A-6, an exemplary multi-piece plunger 10 configured to be slideably advanced through a syringe barrel 112 (shown in FIGS. 5A, 5B, and 6) is illustrated. The plunger 10 includes a head or head portion 12 having an open proximal end 14, a closed distal end 16 having a roof or contact surface configured to contact fluid contained in the syringe barrel 112, and an annular sidewall 18 extending therebetween. Optionally, the head portion 12 can have an outer diameter of about 0.50 inches and a length of about 0.376 inches not including the distal end 16 or tip. The head portion 12 can be formed from a suitable rigid plastic material including, for example, one or more of polyolefins (e.g., PE, PP, and their copolymers), cyclic olefin polymer or copolymer, polyamides (e.g., nylons), polyesters (e.g., PET), polystyrene, polyurethane, polycarbonate, acrylonitrile-butadiene-styrene, fluoropolymers, ionomers, polyacrylates, or any other similar material. The plunger 10 further includes a plurality of annular seals, such as a proximal seal 30 and a distal seal 32, disposed around the sidewall 18 of the head portion 12.

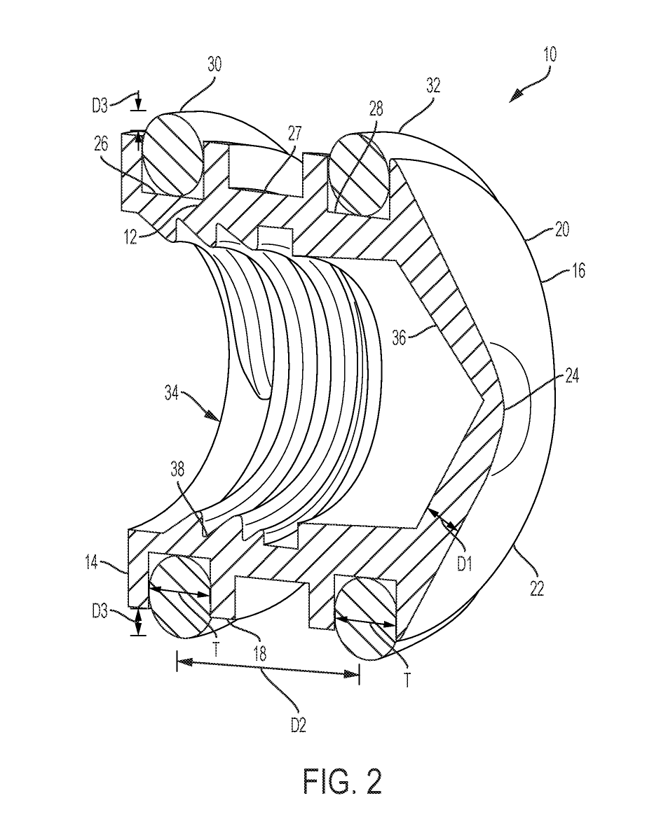

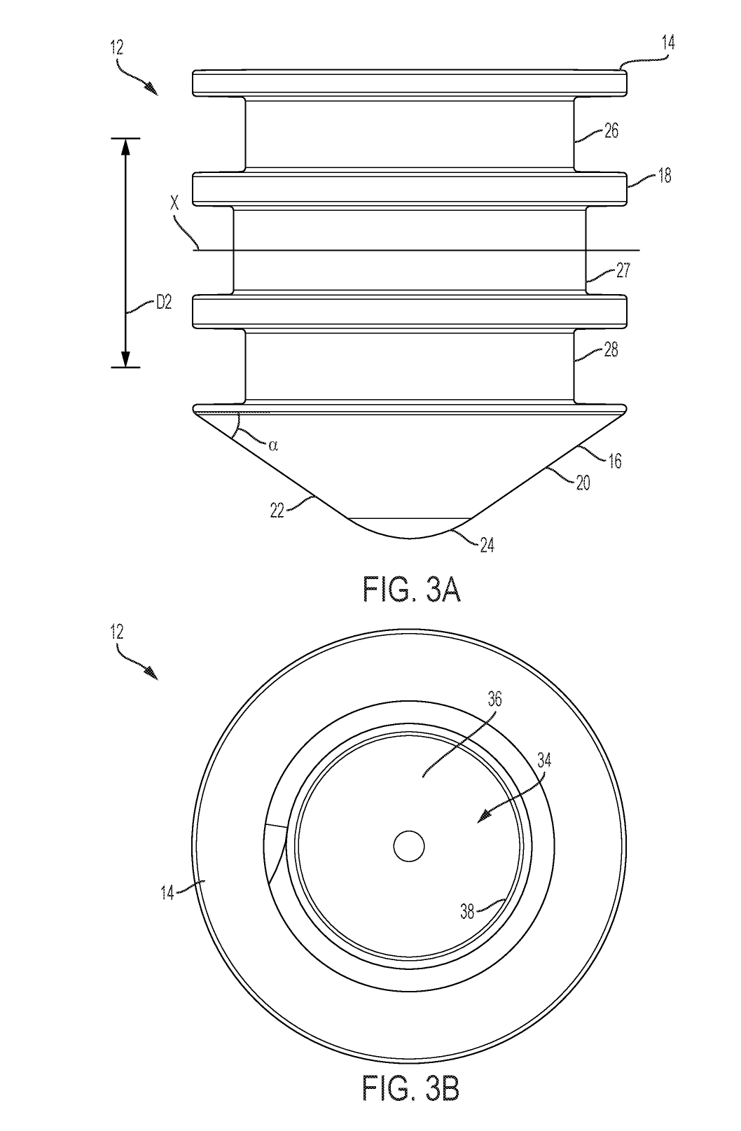

With specific reference to FIGS. 2, 3A, and 3B, in some examples, the closed distal end 16 of the head portion 12 includes a first angled portion 20 and a second angled portion 22, which are both part of a single conical surface that extends toward a tip 24, thereby providing the closed distal end 16 with a substantially conical appearance. As shown in FIG. 3A, the conical angle α can be about 35°, however, various alternative conical angles α are also contemplated herein. Optionally, the conical distal end of the head portion 12 can have a thickness D1 (shown in FIG. 2) of about 0.048 inches. An exemplary geometry of a distal end of a stopper, which can be used for the head portion 12 of the presently disclosed multi-piece plunger, is described in U.S. Pat. No. 7,331,942, entitled “Flush syringe having anti-reflux stopper”, which is hereby incorporated by reference in its entirety. However, this shape of the flexible roof is not to be considered as limiting the present invention as the roof may be flat.

In some examples, the sidewall 18 can define a plurality of annular grooves, such as a proximal groove 26 and a distal groove 28, extending radially inward from the outer surface of the sidewall 18. The grooves 26, 28 can be sized and shaped to receive the annular seals 30, 32 (shown in FIG. 2). In some examples, the sidewall 18 can also define a middle groove 27 disposed between the proximal groove 26 and the distal groove 28. However, the middle groove 27 may not receive a corresponding annular seal. The grooves 26, 27, 28 can have a variety of different shapes including, for example, a substantially square or rectangular cross section with opposing vertical walls and a substantially flat bottom surface, as shown, for example, in FIG. 3A. In other examples, the annular grooves 26, 27, 28 can have a triangular shaped cross section with angled opposing walls or a curved cross section. The annular grooves 26, 28 can also be provided on the sidewall in various arrangements. For example, the annular grooves 26, 28 may be separated by a space or gap, which defines the distance between the annular seals of the multi-piece plunger 10. In some examples, the gap can be about the thickness T of one of the annular seals, as shown in FIG. 2. The distance D2 (shown in FIGS. 2 and 3A) between the annular seals 30, 32 can be about 0.262 inches. As discussed herein, the height/diameter ratio of the seal is selected to impart sufficient stability to the plunger 10 and, in particular, to prevent the plunger 10 from flipping inside out during insertion in a tube, such as an insertion tube used for vented tube placement. In other examples, as discussed herein, the grooves 26, 28 and annular seals 30, 32 can be enlarged, thereby reducing the distance D2 between the seals. In some examples, the grooves 26, 28 are substantially equidistant from a central latitudinal or radial axis X of the plunger head 12. In other examples, the grooves 26, 28 may be disposed closer to the distal end 16 of the plunger head 12 to impart particular sliding characteristics for the plunger 10 and/or prefilled syringe.

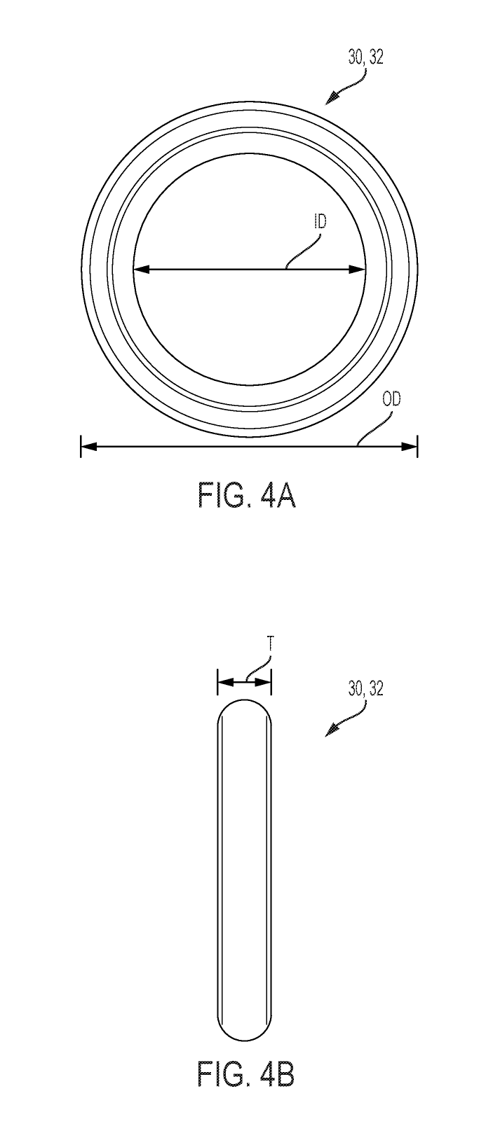

With specific reference to FIG. 2, the annular seals 30, 32 can be mounted in respective annular grooves such that at least a portion of each of the annular seals 30, 32 protrudes radially beyond the outer surface sidewall 18 to form the slideable seal with an inner sidewall of the syringe barrel 112 (shown in FIGS. 5A, 5B, and 6). In this way, the annular seals 30, 32 replace or supplement annular ribs or protrusions found in conventional elastomer or rubber stoppers and plungers. For example, the annular seals 30, 32 may protrude a distance D3 (shown in FIG. 2) beyond the outer surface of the plunger sidewall 18. In some examples, the distance D3 is about one half of the thickness T (shown in FIGS. 2 and 4B) of the annular seal 30, 32. In other examples, the protrusion distance D3 can be less than 30% or less than 20% of the thickness T of the annular seals 30, 32.

In some examples, the head portion 12 can define a plunger rod receiving portion or cavity 34 extending inwardly from the open proximal end 14 of the head portion 12 and being configured to receive a distal end of a plunger rod 114 (shown in FIG. 6). The cavity 34 can define a substantially circular distal surface 36 and a straight or tapered cylindrical sidewall 38 extending between the proximal open end 16 of the head portion 12 and the surface 36. In some examples, the cavity sidewall 38 includes threaded grooves configured to engage corresponding threads of the distal end of the plunger rod 114. In other examples, the distal end of the plunger rod can be mounted into the cavity 34 with a snap fit mechanism or other connector, as is known in the art.

With specific reference to FIGS. 4A and 4B, in some examples, the annular seals 30, 32 are elastomeric O-rings formed from suitable elastomeric materials including, for example, one or more of thermoplastic olefins, styrenic block copolymers, thermoplastic polyurethanes, and thermoplastic polyamides. While the size and shape of the annular seals 30, 32 are largely dependent on the syringe size and volume, the seals 30, 32 can have an inner diameter ID of about 0.410 inches, an outer diameter OD of about 0.590 inches, and a thickness of about 0.090 inches. In some examples, a lubricant, such as silicone or silicone oil, is provided on the surface of each annular seal 30, 32 to improve sliding characteristics. As discussed above, the silicone lubricant can be applied by a tumbling process, in which the annular seals are immersed in a lubricant bath to form a coating on an outer surface of the annular seals 30, 32.

In use, the multi-piece plunger 10, is configured to be inserted into the syringe barrel 112 (shown in FIGS. 5A, 5B, and 6). As shown in FIG. 5A, prior to insertion, the annular seals 30, 32 are in an expanded position having a generally curved outer surface. The outer diameter of the annular seals is selected to be slightly larger than the inner diameter of the syringe barrel 112. Accordingly, when inserted in the syringe barrel 112, the radially outer portion of the annular seals 30, 32 is slightly compressed thereby forming a sufficient seal with the inner surface of the syringe barrel 112. The degree of compression of the annular seals is selected to permit the plunger 10 to easily slide through the barrel 112, but also to provide robust sealing properties, which prevent leaks and/or contamination of fluid contained in the prefilled syringe. The plunger 10 inserted in the syringe barrel 112 with the annular seals 30, 32 in a compressed position is shown, for example, in FIG. 5B.

### Exemplary Syringe Assembly

With reference to FIG. 6, the multi-piece plunger 10 can be used with a syringe assembly 100, such as a prefilled syringe. In addition to the multi-piece plunger 10 and syringe barrel 112 described hereinabove, the assembly 100 includes a plunger rod 114 configured to be removeably coupled to the plunger 10. The plunger rod 114 is an elongated structure extending along longitudinal axis A from a proximal end having a thumb press flange 126 and a distal end including a receiving portion 116. The receiving portion 116 may be configured to be screwed into the cavity 34 (shown in FIGS. 2 and 3B) of the plunger 10 such that threads on the receiving portion 116 of the plunger rod 114 engage corresponding threads on the cavity sidewall 38 (shown in FIG. 2). In some examples, the plunger rod 114 is formed from the same rigid plastic material as the head portion 12 of the plunger 10. For example, the plunger rod 114 can be formed from one or more of polypropylene, polyethylene, cyclic olefin polymer or copolymer, polycarbonate, and polyester.

The plunger rod 114 can further include one or more retaining rings or disks 118 proximal to the receiving portion 116 and configured to engage a corresponding portion of the plunger 10, such as a notch or groove located near the proximal open end 14 of the cavity 34. The engagement between the retaining disk 118 and cavity 34 is configured to prevent a user from inadvertently pulling the plunger rod 114 out of the plunger 10 during aspiration. Specifically, when the disk 118 is engaged to the groove or notch, the plunger 10 and plunger rod 114 are locked together and cannot be disconnected merely by pulling the plunger rod 114 and/or plunger 10 in a proximal direction P.

The assembly 100 further includes the syringe barrel 112. The syringe barrel 112 may be formed of glass, or may be injection molded from thermoplastic material, such as polypropylene and polyethylene, according to techniques known to those of ordinary skill in the art. However, it is to be appreciated that syringe barrel 112 may be made from other suitable materials and according to other applicable techniques as well. Often the material is transparent or translucent so that position of the plunger 10 and/or amount of fluid remaining in the barrel 112 can be easily ascertained. The syringe barrel 112 is generally a substantially cylindrical tubular structure, though syringe barrels having an oval, square, or rectangular cross section may also be used within the scope of the present disclosure. In some examples, the syringe barrel 112 includes a distal or frontal end 120, which includes an outlet opening, such as a nozzle 122. The nozzle 122 can include a mechanism for attachment of a needle cannula or a separate medical device (such as a catheter). For example, the attachment mechanism can be a luer connector, threaded connector, snap fit connector, or similar connection mechanism. The syringe barrel 112 also includes an open proximal or rearward end 124 for receiving the multi-piece plunger 10 and plunger rod 114 assembly. In some examples, the rearward end 124 can include a flange 128 positioned to permit a user to grasp the rearward end of the syringe barrel 112 with his/her forefingers when advancing the plunger rod 114 through the barrel 112.

In use, a user may obtain an empty syringe assembly 100 in which the multi-piece plunger 10 is disposed adjacent to the distal end 120 of the syringe barrel 112. The user can draw fluid into the syringe barrel 112 by moving the plunger 10 in a proximal direction P (e.g., a process often referred to as aspiration). Once a desired amount of fluid is drawn into the syringe barrel 112, the user can advance the plunger 10 through the barrel 112 by pushing on a thumb-press flange 126 of the plunger rod 114 to advance the plunger 10 through the barrel in a distal direction D. Advancing the plunger through the barrel 112 causes fluid contained therein to pass through the nozzle 122 and, in some cases, through a needle cannula or another fluid conducting structure, such as medical tubing, for administration to a patient. In the case of a prefilled syringe, the syringe assembly 100 is often provided from the manufacturer filled with a single dose of fluid to be administered to a patient. In one configuration, the user attaches the plunger rod 114 to the multi-piece plunger 10 by, for example, inserting the receiving portion 116 of the rod 114 into the plunger cavity 34, such that the threads or another connection mechanism lock the plunger 10 and plunger rod 114 together. In another configuration, the syringe may be provided in a pre-assembled configuration in which the syringe is provided with the plunger rod 114 already attached to the multiple piece plunger 10. The user then performs the injection by pushing the plunger 10 through the barrel in the distal direction D.

### Alternative Exemplary Multi-Piece Plungers

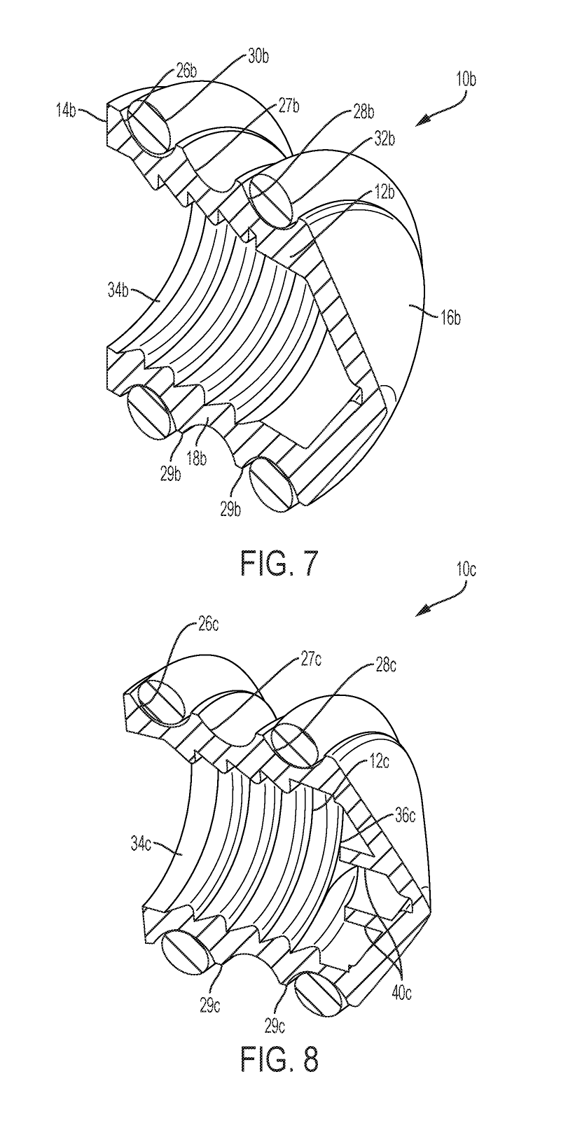

With reference to FIG. 7, another example of a multi-piece plunger 10b configured to be advanced through a syringe barrel to expel fluid therefrom is illustrated. As in previously described examples, the multi-piece plunger 10b includes a head or head portion 12b having an open proximal end 14b, a closed distal end 16b having a roof or angled contact surface configured to contact fluid contained in the syringe barrel, and an annular sidewall 18b extending therebetween. The sidewall 18b defines a plurality of annular grooves, such as a proximal groove 26b and a distal groove 28b, extending radially inward from the outer surface of the sidewall 18b. Each of the grooves 26b, 28b can be sized and shaped to receive an elastomeric annular seal, such as a proximal annular seal 30b and a distal annular seal 32b. Unlike in previously described examples in which the annular grooves had a square or rectangular cross section, the annular grooves 26b, 28b can define a substantially concavely curved surface, having a curvature substantially similar to the curvature of the annular seals 30b, 32b. The head portion 12b may also include a central groove 27b defining a curved surface; however, an annular seal is not inserted in the central groove 27b. The grooves 26b, 27b, 28b are separated by a small flat annular surface 29b. In other examples of the head portion 12b, the flat annular surface 29b can be removed and/or replaced by a pointed ridge between each of the one or more grooves 26b, 27b, 28b.

As in previously described examples, the head portion 12b of the plunger 10b defines a cavity 34b extending inwardly from the open proximal end 14b thereof. The cavity 34b is configured to receive the receiving portion of a plunger rod to removeably connect the plunger 10b to the plunger rod.

With reference to FIG. 8, another exemplary multi-piece plunger, generally labelled 10c, is illustrated. As with the embodiment of FIG. 7, the plunger 10c includes a head portion 12c with annular grooves 26c, 27c, 28c defining curved surfaces and separated by annular flat surfaces 29c. The multi-piece plunger 10c, shown in FIG. 8, also includes a cavity 34c. However, unlike in previously described examples, a distal end 36c of the cavity 34c includes one or more longitudinal supports 40c extending toward the roof or angled contact surface of the plunger 10c. The supports 40c can be arranged in various configurations including, for example, x-shaped or cross patterns, in which a first support is orthogonal to a second support, and/or in a circular or cylindrical orientation. In other examples, two or more supports 40c can be arranged parallel to one another. The supports 40c are positioned to prevent or reduce deflection of the roof or angled contact surface of the plunger 10c, which can occur when the plunger 10c contacts fluid contained in the syringe barrel. The supports 40c can also prevent or reduce syringe reflux by limiting deformation of the angled contact surface, thereby reducing the distance that the plunger 10c bounces in a proximal direction after contacting the distal end of the syringe barrel.

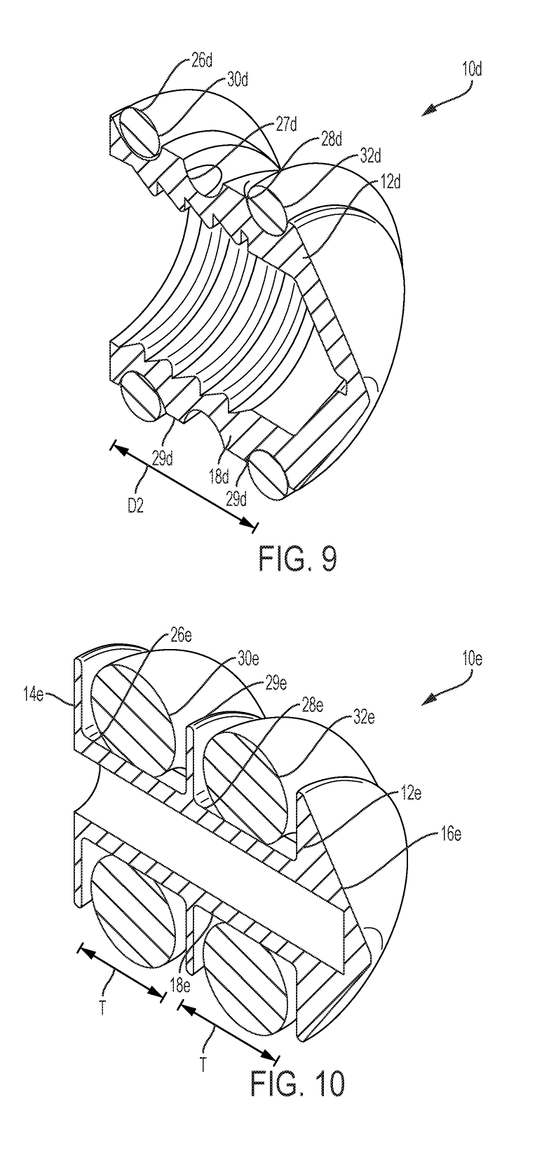

With reference to FIG. 9, another exemplary multi-piece plunger, generally labelled 10d, configured to be advanced through a syringe barrel to expel fluid therefrom is illustrated. As in previously described examples, the plunger 10d includes a head or head portion 12d including a sidewall 18d, which defines a plurality of annular grooves, such as a proximal groove 26d and a distal groove 28d, extending radially inward from the outer surface of the sidewall 18d. Each of the grooves 26d, 28d can be sized and shaped to receive an elastomeric annular seal, such as a proximal annular seal 30d and a distal annular seal 32d. The annular grooves 26d, 28d can define a substantially concavely curved surface, having a curvature substantially similar to the curvature of the annular seals 30d, 32d. The head portion 12d also includes a central groove 27d defining a curved surface; however, an annular seal is not inserted in the central groove 27d. Unlike in previously described examples, which included only small annular surfaces or pointed ridges between adjacent grooves, as shown in FIG. 9, the grooves 26d, 27d, 28d are separated by enlarged flat annular surfaces 29d. Increasing the width of the flat surfaces 29d between the grooves 26d, 27d, 28d increases the distance between the annular seals 30d, 32d, thereby increasing the total length D2 of the seal compared with previously described examples. While not intending to be bound by theory, it is believed that increasing the total width of the seal improves stability of the multi-piece plunger even though the surface area of contact between the annular seals 30d, 32d and syringe barrel is not increased.

With reference to FIG. 10, another example of a multi-piece plunger 10e configured to be advanced through a syringe barrel to expel fluid therefrom and including thicker annular seals is illustrated. As in previously described examples, the multi-piece plunger 10e includes a head or head portion 12e having an open proximal end 14e, a closed distal end 16e having a roof or angled contact surface configured to contact fluid contained in the syringe barrel, and an annular sidewall 18e extending therebetween. The sidewall 18e defines a plurality of annular grooves, such as a proximal groove 26e and a distal groove 28e, extending radially inward from the outer surface of the sidewall 18e. The grooves 26e, 28e have a substantially square-shaped or rectangular cross section with opposing vertical walls and a flat bottom surface. Unlike in previously discussed examples, the head portion 12e does not include a central groove between the grooves 26e, 28e. Each of the grooves 26e, 28e is sized and shaped to receive an enlarged elastomeric annular seal, such as a proximal annular seal 30e and a distal annular seal 32e. In some examples, the seals 30e, 32e have a thickness T of about 0.195 inches, which is about double the thickness of the annular seals show in FIGS. 1-9. The grooves 30e, 32e are separated by a small flat annular surface 29e. The thicker seals 30e, 32e have a larger area of contact with syringe barrel compared to previously described examples. Increasing the area of contact between the seals 30e, 32e and the syringe barrel increases the stability and fluid-tight properties of the seal. However, increasing the surface area of the seal may increase the force needed to slide the plunger 10e through the syringe barrel and/or may increase the amount of lubricant required to impart suitable sliding characteristics to the plunger 10e.

### Exemplary Two-Piece Plungers

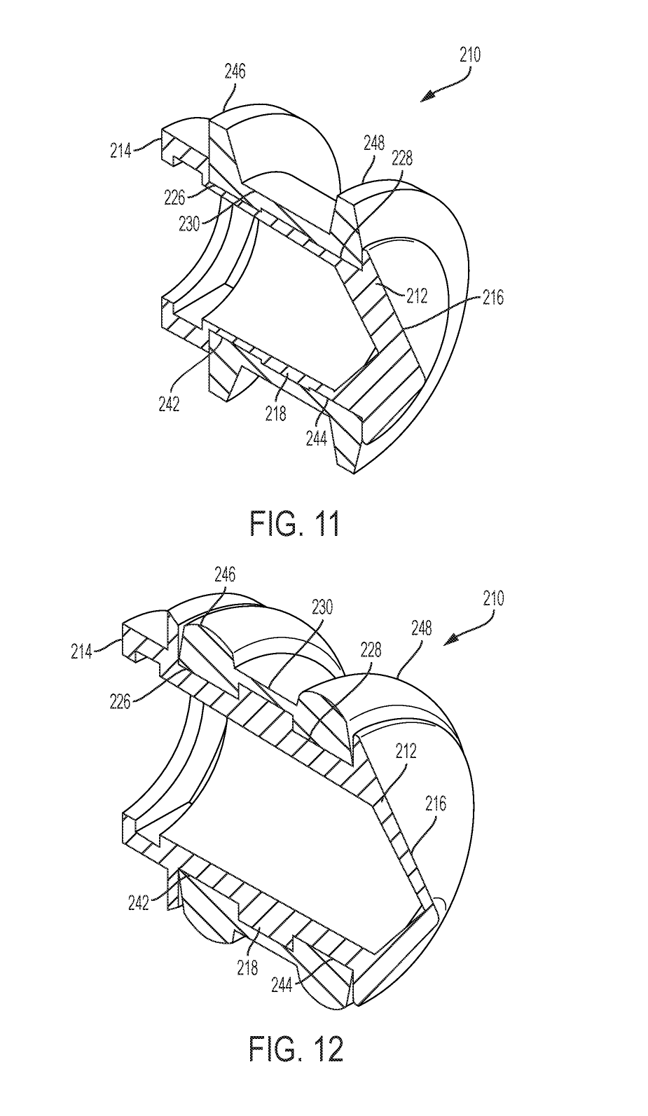

According to another aspect of the disclosure, examples of two-piece plungers 210, 210b are illustrated in FIGS. 11-13. With specific reference to FIGS. 11 and 12, the two-piece plunger 210 includes a head or head portion 212 having an open proximal end 214, a closed distal end 216 having a roof or contact surface configured to contact fluid contained in a syringe barrel, and an annular sidewall 218 extending therebetween. The head portion 212 can be formed from a suitable rigid polymer material including, for example, one or more of polyolefins (e.g., PE, PP, and their copolymers), cyclic olefin polymer or copolymer, polyamides (e.g., nylons), polyesters (e.g., PET), polystyrene, polyurethane, polycarbonate, acrylonitrile-butadiene-styrene, fluoropolymers, ionomers, polyacrylates, or any other similar material.

The plunger 210 further includes a single annular seal 230 disposed about the sidewall 218. The annular seal 230 can be formed from an elastomeric material including, for example, one or more of thermoplastic olefins, styrenic block copolymers, thermoplastic polyurethanes, and thermoplastic polyamides. The head portion 212 and annular seal 230 can be made by any suitable method including injection molding by two-shot molding. In other examples, the annular seal 230 can be overmolded on a molded head or head portion 212. In other examples, the annular seal 230 can be formed separately. In that case, the plunger 210 can be assembled by pulling the annular seal 230 over the head portion 212.

In some examples, the plunger head portion 212 can include a number of shallow annular grooves, such as a proximal groove 226 and a distal groove 228. Portions of the annular seal 230, such as a proximal protrusion 242 and a distal protrusion 244, can be configured to be inserted in the respective grooves 226, 228. The annular seal 230 also includes at least two radially protruding ribs or ridges, such as a proximal ridge 246 and a distal ridge 248 extending radially outward beyond the sidewall 218 of the head portion 212. The ridges 246, 248 are configured to contact an inner surface of the syringe barrel to form a suitable seal therewith. As shown in FIG. 11, the ridges 246, 248 can have a polygonal or tetrahedral cross section with a substantially flat annular outer surface configured to contact the syringe barrel. In other examples, the ridges or ribs can have a square-shaped cross section, a triangular cross section, or can be curved. For example, a two-piece plunger 210 having radially extending ribs or ridges 246, 248 with a curved outer surface is shown in FIG. 12.

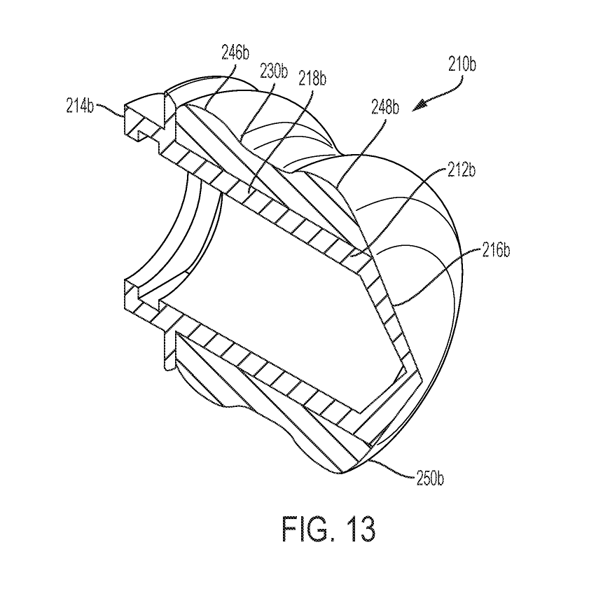

With specific reference to FIG. 13, another example of two-piece plungers 210b is illustrated. As in the previously described examples, the two-piece plunger 210b includes a head or head portion 212b having an open proximal end 214b, a closed distal end 216b having a roof or conical contact surface configured to contact fluid contained in a syringe barrel, and an annular sidewall 218b extending therebetween. The plunger 210b further includes a single annular elastomeric seal 230b disposed about the sidewall 218b. The annular seal 230b includes at least two radially protruding ribs or ridges, such as a proximal annular ridge 246b and a distal ridge 248b extending radially outward beyond the sidewall 218b of the head portion 212b. As shown in FIG. 13, the ridges 246b, 248b may have a curved outer surface and a semi-circular cross section. The ridges 246b, 248b can be tapered or angled, thereby providing a substantially continuous sidewall surface without vertical walls or other discontinuities. In some examples, a distal portion 250b of the annular seal 230b can extend around the conical contact surface of the head portion 212b at the distal end 216b thereof. The distal portion 250b of the annular seal 230b can be angled or tapered in the same manner as the conical contact surface of the head portion 212b, thereby creating a two-piece plunger 210b having a distal contact surface formed from both the head portion 212b and the annular seal 230b.

### Exemplary Manufacturing Method

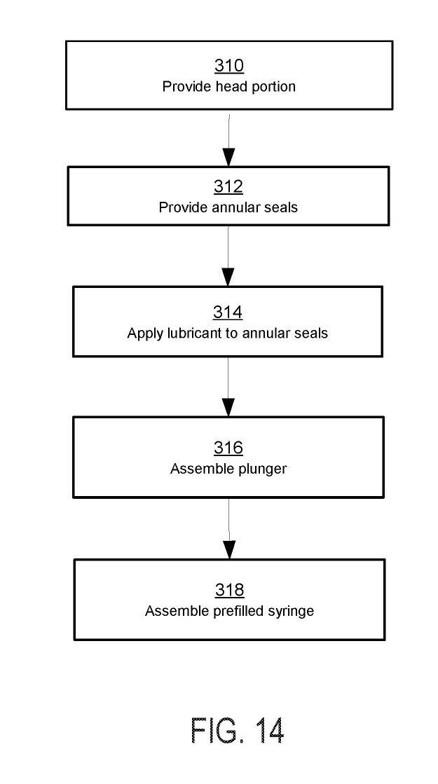

With reference to FIG. 14, steps for manufacture of a multi-piece plunger including a head portion and one or more annular seals, such as the multi-piece plungers shown in FIGS. 1-13, are discussed. As shown at box 310, the head portion of the plunger is provided. The head portion can include, for example, a proximal end, a distal end, and an annular sidewall extending therebetween. The annular sidewall of the head portion can include structures for receiving elastomeric annular seals, such as the annular grooves discussed hereinabove. The head portion can be molded by conventional molding processes, as are known in the art. For example, the head portion can be molded by an injection molding, in which fluid polymer material is injected into a mold and cured to form the head portion. As discussed herein, the head portion can be formed from a suitable rigid polymer material including, for example, one or more of polyolefins (e.g., PE, PP, and their copolymers), cyclic olefin polymer or copolymer, polyamides (e.g., nylons), polyesters (e.g., PET), polystyrene, polyurethane, polycarbonate, acrylonitrile-butadiene-styrene, fluoropolymers, ionomers, polyacrylates, or any other similar material.

As shown at box 312, the method further includes providing the one or more annular seals. As discussed herein, the annular seals can be elastomeric structures, such as O-rings, formed from a suitable elastomeric material. In some examples, the head portion and seals can be formed substantially simultaneously, as occurs during two-shot molding processes. In some examples, providing the annular seals can include overmolding the seals to the head portion. In other examples, the annular seals can be formed separately from the head portion. For example, the one or more annular seals may be formed by injection molding in individual molds or may be pressed or stamped from sheets of elastomeric material. In some examples, as shown at box 314, a lubricant, such as silicone oil, can be applied to the annular seals to improve sliding characteristics of the seals through a syringe barrel. For example, prior to being attached to the head portion, the annular seals can be tumbled or immersed in a lubricant bath to coat the outer surface thereof with the lubricant. As shown at box 316, after the head portion and annular seals are prepared, the plunger can be assembled by attaching the annular seals to the head portion. For example, assembly can include manually or automatically sliding the annular seals over the head portion and allowing the seals to contract into the respective annular grooves on the head portion.

As shown at box 318, a prefilled syringe can be assembled by filling a syringe barrel with a fluid, such as a fluid containing a therapeutic agent, and inserting the assembled plunger into the syringe barrel. For example, as discussed herein, a prefilled syringe can be produced by filling the syringe barrel with fluid through the open proximal end thereof. After the syringe barrel is filled, the multi-piece plunger can be inserted through the open proximal end of the barrel by a vented placement method as is known in the art. In other examples, the plunger can be inserted by a vacuum stoppering method.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of invention which is to be given the full breadth of the claims appended and any and all equivalents thereof.

The invention claimed is:

1. A plunger configured to be slidably advanced through a syringe barrel to expel fluid therefrom, the plunger comprising:
a head portion comprising a proximal end, a distal end, and an annular sidewall extending therebetween, wherein the sidewall defines a first annular groove and a second annular groove;
a first annular seal disposed within the first annular groove; and
a second annular seal disposed within the second annular groove, such that at least a portion of each of the first and second annular seals protrudes radially beyond an outer surface of the sidewall to form a slideable seal with an inner sidewall of the syringe barrel.

2. The plunger of claim 1, wherein the head portion comprises a first material, and the annular seals comprise a second material that is different from the first material.

3. The plunger of claim 2, wherein the first material comprises one or more of polypropylene, polyethylene, cyclic olefin polymer or copolymer, polycarbonate, and polyester, and wherein the second material comprises one or more of a thermoset rubber and a thermoplastic elastomer.

4. The plunger of claim 3, wherein the thermoset rubber comprises one or more of butyl rubber, styrene-butadiene rubber, and poly-isoprene rubber, and wherein the thermoplastic elastomer comprises one or more of thermoplastic olefins, styrenic block copolymers, thermoplastic polyurethanes, and thermoplastic polyamides.

5. The plunger of claim 1, wherein the annular seals comprise a lubricant disposed on an outer surface thereof.

6. The plunger of claim 5, wherein the lubricant comprises silicone.

7. The plunger of claim 5, wherein the head portion of the plunger is lubricant free.

8. The plunger of claim 1, wherein the head portion defines a cavity configured to receive a receiving portion of a plunger rod, the cavity extending inward from the proximal end of the plunger head.

9. The plunger of claim 8, wherein the cavity comprises a sidewall defining a plurality of threaded grooves configured to engage corresponding threads of the distal end of the plunger rod.

10. The plunger of claim 8, wherein the head portion is configured to receive a portion plunger rod in a snap-fit configuration.

11. The plunger of claim 8, where the cavity comprises one or more longitudinal supports extending in a distal direction from a distal end thereof, the longitudinal supports being configured to support the closed distal end of the head portion.

12. The plunger of claim 1, wherein the distal end of the head portion comprises a conical contact surface.

13. The plunger of claim 1, wherein the at least two annular grooves are spaced apart in a longitudinal direction by a gap having a length that is greater than or equal to a thickness of the annular seals.

14. The plunger of claim 1, wherein the at least two annular grooves comprise a concavely curved surface having a curvature that corresponds to the curvature of the annular seals.

15. A syringe comprising:
a substantially cylindrical barrel having an open proximal end, a distal end comprising a nozzle, and a sidewall extending therebetween; and
a plunger slideably disposed within the syringe barrel and configured to be advanced through the barrel in a distal direction to expel fluid through the nozzle, wherein the plunger comprises:
a head portion comprising a proximal end, a distal end, and an annular sidewall extending therebetween, wherein the sidewall defines at least two annular grooves; and
annular seals disposed within each of the at least two annular grooves, such that at least a portion of each of the annular seals protrudes radially beyond the sidewall to form a slideable seal with an inner sidewall of the syringe barrel.

16. The syringe of claim 15, further comprising a plunger rod, and wherein a distal end of the plunger rod is removeably inserted in a cavity extending inward from the proximal end of the plunger head.

17. The syringe of claim 16, wherein the distal end of the plunger rod comprises threaded grooves configured to engage corresponding threaded grooves on an inner sidewall of the cavity.

18. The syringe of claim 16, wherein the plunger rod and the head portion comprise a first material, and wherein the annular seals comprise a second material, the second material being different than the first material.

19. The syringe of claim 18, wherein the first material comprises one or more of polypropylene, polyethylene, cyclic olefin polymer or copolymer, polycarbonate, and polyester, and wherein the second material comprises one or more of butyl rubber, thermoplastic olefins, styrenic block copolymers, thermoplastic polyurethanes, and thermoplastic polyamides.

20. A method of manufacture of a prefilled syringe, comprising:
molding a head portion of a multi-piece plunger, wherein the head portion comprises a proximal end, a distal end, and an annular sidewall extending therebetween;
providing at least one annular seal around at least a portion of the sidewall of the plunger, thereby forming the multi-piece plunger; and
inserting the multi-piece plunger into a syringe barrel thereby forming a prefilled syringe, wherein, when inserted in the barrel, at least a portion of the at least one annular seal contacts an inner sidewall of the syringe barrel to form a slideable seal therewith.

21. The method of claim 20, wherein the head portion comprises a first material, and the at least one annular seal comprises a second material that is different from the first material.

22. The method of claim 21, wherein the first material comprises one or more of polypropylene, polyethylene, cyclic olefin polymer or copolymer, polycarbonate, and polyester, and wherein the second material comprises one or more of butyl rubber, thermoplastic olefins, styrenic block copolymers, thermoplastic polyurethanes, and thermoplastic polyamides.

23. The method of claim 20, wherein providing the at least one annular seal comprises forming the seal by overmolding the seal on the head portion.

24. The method of claim 20, wherein providing the at least one annular seal comprises molding the at least one annular seal from an elastomeric material different from the material used to mold the head portion, and assembling the plunger by inserting the at least one annular seal in a groove on the sidewall of the head portion.

25. The method of claim 20, further comprising filling the syringe barrel with a fluid prior to insertion of the multi-piece plunger.

26. The method of claim 20, wherein inserting the plunger into the syringe barrel comprises inserting the plunger by a vented placement method.

27. A plunger configured to be slideably advanced through a syringe barrel to expel fluid therefrom, the plunger comprising:
a head portion comprising a proximal end, a distal end, and an annular sidewall extending therebetween; and
an annular seal disposed about at least a portion of the sidewall,
wherein the annular seal comprises at least two annular ribs protruding radially outward from the seal and configured to form a slideable seal with an inner sidewall of the syringe barrel.

28. The plunger of claim 27, wherein the head portion comprises a first material, and the annular seal comprises a second material that is different from the first material.

29. The plunger of claim 28, wherein the first material comprises one or more of polypropylene, polyethylene, cyclic olefin polymer or copolymer, polycarbonate, and polyester, and wherein the second material comprises one or more of butyl rubber, thermoplastic olefins, styrenic block copolymers, thermoplastic polyurethanes, and thermoplastic polyamides.

30. The plunger of claim 27, wherein the head portion comprises at least two annular grooves on the sidewall thereof, and wherein a portion of the annular seal is disposed within each of the at least two annular grooves.

31. The plunger of claim 27, wherein the at least two annular ribs have a polygonal shaped cross section.

32. The plunger of claim 27, wherein the at least two annular ribs are convexly curved.
